(19) United States
(12) Patent Application Publication
(10) Pub. No.: US 2025/0276291 A1
(43) Pub. Date: Sep. 4, 2025

(54) FLUID FILTRATION APPARATUS AND RELATED METHODS

(71) Applicant: STEMCELL TECHNOLOGIES CANADA INC., Vancouver (CA)

(72) Inventors: Mark WILLIAMSON, Vancouver (CA); Kevin CHEN, Vancouver (CA); Benjamin STARKEY, Vancouver (CA); Cole STEVENS, Vancouver (CA)

(21) Appl. No.: 19/210,218

(22) Filed: May 16, 2025

Related U.S. Application Data
(63) Continuation of application No. PCT/CA2023/051550, filed on Nov. 17, 2023.
(60) Provisional application No. 63/384,395, filed on Nov. 18, 2022.

(51) Int. Cl. B01D 63/16; A61M 1/16; A61M 1/34; B01D 63/06

(52) U.S. Cl. CPC B01D 63/16; A61M 1/1621; A61M 1/3496; B01D 63/061; A61M 2205/0272; A61M 2207/00; B01D 2313/10

(57) ABSTRACT

This disclosure relates to a fluid filtration apparatus, and to methods of making and using a fluid filtration apparatus of this disclosure. Fluid filtration apparatus includes a frame having a porous member secured thereto, and the frame is rotatably supported within a housing. Fluid, and particulates therein, are introduced via an inlet and components of the fluid may be filtered through the porous member to yield a filtrate (and particulates having passed through the porous member) and a retentate (and particulates not having passed through the porous member). Methods of manufacturing (and assembling) and methods of using a fluid filtration apparatus of this disclosure are also described.

### CROSS REFERENCE TO RELATED APPLICATION

This application is a continuation of Patent Cooperation Treaty (PCT) application No. PCT/CA2023/051550, having an international filing date of 17 Nov. 2023, which in turns claims the benefit of 35 USC § 119 in respect of U.S. Provisional Patent Application No. 63/384,395 filed Nov. 18, 2022. All of the applications in this paragraph are hereby incorporated herein by reference.

### TECHNICAL FIELD

This disclosure relates to a fluid filtration apparatus, such as for blood or plasma processing systems and methods. More particularly, the disclosure relates to fluid filtration apparatus and systems, and to methods for manufacturing or using a fluid filtration apparatus, Fluid filtration apparatus of this disclosure may be included in an automated and closed system for separating or enriching target cells, such as for cell therapy applications.

### BACKGROUND

Process automation of cell manufacturing and/or separation is important for the widespread adoption of cell therapy technologies. It is highly desirable that automated cell processing platforms limit the number of times, or steps, in which a cell population is manipulated and/or exposed to the external environment so as to limit contamination and other problems.

Spinning filter separators are currently used to separate specific cellular blood components (e.g., red blood cells, white blood cells, and platelets) from specific particulates in whole blood, before use in downstream applications. The targeted cell product (e.g., red blood cells, white blood cells, or platelets) may be in a suspension that includes plasma and/or some other supernatant. As such, it is sometimes desirable to “wash” the cellular suspension (typically with a physiologic buffer) to remove the plasma/supernatant, as well as any non-target cellular material, prior to reinfusion.

As an example, cell processing may be necessary during the manufacture of CAR T cell-based cell therapies, wherein CD4+ and CD8+ T-cells are isolated from a leukopak sample before T-cell activation and genetic modification, CAR T cell expansion, and CAR T cell reinfusion into a patient. As the currently available CAR T cell therapies are typically customized for each individual patient, the need for efficient and automated systems is heightened.

Accordingly, improved fluid filtration apparatus and related processes are highly desirable for: automated and/or closed systems; fast and easy processing protocols (e.g., concentrating or washing) for various applications, such as the CAR T-cell therapies.

### SUMMARY

The present disclosure relates to a fluid filtration apparatus and system for automated processing of a blood product for cell therapy applications. In one aspect, the present disclosure is directed to a method for manufacturing the fluid filtration apparatus.

In an aspect of this disclosure are provided fluid filtration apparatus, as may be used in systems for processing blood or apheresis samples.

In one embodiment, fluid filtration apparatus of this disclosure comprise (i) a housing defining a lumen bounded by at least one sidewall, the housing having an upper end and an opposed lower end, (ii) an inlet and a first outlet through the housing, each of the inlet and the first outlet in fluid communication with the lumen, (iii) a second outlet through the housing, (iv) a frame rotatably supported within the lumen and rotatable relative to the housing, (v) at least one bore through the frame, the at least one bore in fluid communication with the second outlet, (vi) a first porous member secured to or about the frame.

In one embodiment, the frame and/or the housing are cylindrical or substantially cylindrical.

In one embodiment, a lumen-facing surface of the at least one sidewall tapers from the upper end of the housing to the lower end of the housing by about 5 degrees or less, about 3 degrees or less, or about 1 degree±0.5 degrees.

A fluid filtration apparatus may further comprise an upper assembly and a lower assembly that cooperate respectively with the upper end of the housing and the lower end of the housing to rotatably support the frame in the lumen. In one embodiment, the upper assembly and the lower assembly define a longitudinal axis through the lumen, and the frame rotates about the longitudinal axis.

A fluid filtration apparatus may further comprise a baffle configured in at least the non-tangential inlet, and preferably configured at an opening of the inlet into the lumen.

A fluid filtration apparatus may further comprise a magnetically-responsive member fixedly arranged in the frame, preferably in an upper end of the frame. In one embodiment, the magnetically-responsive member and the frame are mated via a pair of complementary features comprising at least one straight edge.

In one embodiment, the first porous member is secured to the frame by a pressure sensitive adhesive. In one embodiment, the first porous member is a track etch membrane. In one embodiment, the porous membrane has a pore density of between about 1×10⁵ pores/cm² to 1×10⁶ pores/cm².

A fluid filtration apparatus may further comprise a second porous member secured to or about the frame. In one embodiment, the second porous member is secured to or about the frame by a pressure sensitive adhesive. In one embodiment, the second porous member is a track etch membrane.

A fluid filtration apparatus may further comprise a plurality of spaced-apart latitudinal grooves in a porous member facing surface of the frame. In one embodiment, a fluid filtration may further comprise a plurality of spaced-apart longitudinal grooves in a porous member facing surface of the frame, wherein the plurality of spaced-apart longitudinal grooves interconnect the plurality of spaced-apart latitudinal grooves. In one embodiment, the plurality of spaced-apart longitudinal grooves and the plurality of spaced-apart latitudinal grooves cooperate to receive a filtrate having passed through the porous member and shuttle the filtrate toward the second outlet via the at least one bore in the frame.

In one embodiment, a width of the plurality of spaced apart latitudinal and/or longitudinal grooves are equal. In one embodiment, a width of the plurality of spaced apart latitudinal and/or longitudinal grooves are not equal. In one embodiment, the plurality of spaced-apart latitudinal grooves and/or the plurality of spaced-apart longitudinal grooves are larger in size at the top compared to the bottom of the frame.

In one embodiment, fluid filtration apparatus of this disclosure comprise (i) a housing defining a lumen bounded by at least one sidewall, (ii) an inlet and an outlet through the housing, each of the inlet and the outlet in fluid communication with the lumen, (iii) a waste port through the housing, (iv) a frame supported within the lumen in a relative rotational relationship with the housing, the frame having at least one bore therethrough, the at least one bore in fluid communication with the waste port, (v) a first porous membrane secured to or about the frame, (vi) an upper bearinged assembly rotatably connecting the frame at an upper end thereof and an upper end of the housing, and (vii) a lower assembly rotatably connecting the frame at a lower end thereof and a lower end of the housing.

In one embodiment, the inlet and the first outlet is positioned intermediate the upper assembly and the lower end of the housing.

In one embodiment, a bore through the inlet is non-tangential to the at least one sidewall. In one embodiment, a baffle may be configured in the bore.

In one embodiment, the upper (bearinged) assembly and the lower assembly define a longitudinal axis through the lumen, and the frame rotates about the longitudinal axis thereof.

In one embodiment, magnetically-responsive member is positioned within the upper (bearinged) assembly. In one embodiment, fluid filtration apparatus of this disclosure may further comprise an aperture having at least two substantially straight edges through the magnetically-responsive member. In one embodiment, fluid filtration apparatus of this disclosure may further comprise a torque imparting member within the upper (bearinged) assembly that is connected to the aperture.

In another aspect of this disclosure are provided methods of manufacturing fluid filtration apparatus, as may be used in automated systems for processing blood or apheresis samples.

In one embodiment, a method of manufacturing a fluid filtration apparatus (as may be described herein) may comprise (a) molding a housing having i) a lumen bounded by at least one sidewall that defines a longitudinal axis, ii) an inlet, iii) a first outlet, and iv) a second outlet, each of the inlet, the first outlet, and the second outlet in fluid communication with the lumen, (b) molding a frame, (c) attaching a porous membrane to or about the frame by a pressure sensitive adhesive, and (d) securing the frame within the lumen, the frame rotatable within the lumen about an axis of rotation (which may be coincident with the longitudinal axis).

In one embodiment, a method of manufacturing a fluid filtration apparatus of this disclosure may further comprise configuring at least one baffle in the housing, and preferably in one or both of the inlet and the outlet. In one embodiment, a baffle is configured at an opening of the inlet (and optionally the first inlet) into the lumen.

In one embodiment, a method of manufacturing a fluid filtration apparatus of this disclosure may further comprise applying a pressure sensitive adhesive to one or more frame-contacting faces of the porous member.

In one embodiment, a method of manufacturing a fluid filtration apparatus of this disclosure may further comprise applying a pressure sensitive adhesive to the frame, corresponding to the dimensions of a frame-facing side of the porous member.

In one embodiment, a method of manufacturing a fluid filtration apparatus of this disclosure may further comprise applying the porous member to the frame before inserting the frame in the lumen by rotating the frame about the axis of rotation to set the porous member on the frame.

In one embodiment, a method of manufacturing a fluid filtration apparatus of this disclosure may further comprise applying pressure against the porous member and the frame to secure the porous member thereto.

In one embodiment, one or both of the housing and the frame is injection molded. In one embodiment, one or both of the frame and the housing are molded of a polymer.

In one embodiment, the frame is the frame is rotatably supported in the lumen between an upper end of the housing and a lower end of the housing by an upper assembly and a lower assembly that cooperate respectively with the upper end of the housing and the lower end of the housing.

In one embodiment, a lumen-facing surface of the at least one sidewall tapers from the upper end of the housing to the lower end of the housing by about 5 degrees or less, about 3 degrees or less, or about 1 degree±0.5 degrees.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows an exploded perspective view of the fluid filtration apparatus, according to an exemplary embodiment.

FIG. 2 shows a perspective view of an assembled fluid filtration apparatus, according to an exemplary embodiment.

FIG. 3 shows a cross-sectional, bottom view of a fluid filtration apparatus showing the structural configuration of an inlet and an outlet through housing when viewed from the direction of the arrow A in FIG. 2.

FIG. 4 shows a longitudinal cross-section of an upper portion of a fluid filtration apparatus showing the structural interaction between an upper (bearinged) assembly, a frame, and a housing, according to an exemplary embodiment.

FIG. 5 shows a cross section of the upper (bearinged) assembly when viewed from the direction of the arrow B in FIG. 2.

FIG. 6 shows a longitudinal cross-section of a lower portion of the fluid filtration apparatus, according to an exemplary embodiment. Grey arrows denote the flow of fluid (and particulate matter that may be suspended therein) toward the porous membrane, and black arrows denote the flow of a filtrate (and particulate matter that may be suspended therein) toward and through a second outlet

FIG. 7 shows a longitudinal cross section of a lower portion of a fluid filtration apparatus having been filled with a fluid, according to an exemplary embodiment. An air bubble or pocket is formed at lower assembly as the frame rotates and the fluid moves under centrifugal forces away from an axis of rotation of the frame.

### DETAILED DESCRIPTION

This disclosure relates to spinning filter apparatus, which may be used in methods of separating particulate matter from a fluid or size-based fractionation of particulates in a fluid. Fluid processed by apparatus of this disclosure are not particularly limited, provided they contain or comprise particulate matter. In one embodiment, the fluid is a biological sample, such as blood, plasma, urine, or the like.

### Apparatus

In one aspect, this disclosure relates to spinning membrane/filter apparatus (e.g. a fluid filtration apparatus).

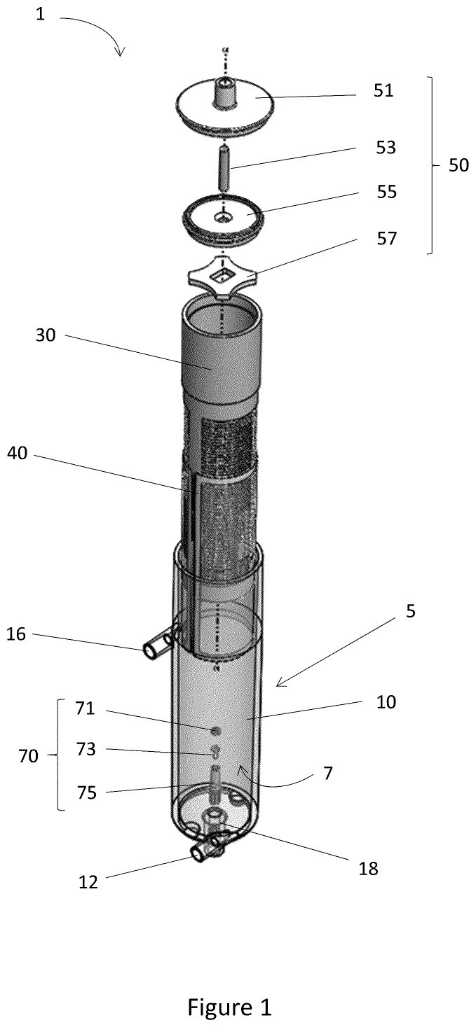

Referring to FIGS. 1 through 7, there is illustrated an embodiment of a fluid filtration apparatus 1. Fluid filtration apparatus 1 comprises a housing 5 defining a lumen 7 bounded by at least one sidewall 10. In one embodiment, housing 5 comprises an upper end and an opposed lower end connected by at least one sidewall 10, the direction (or length) of the at least one sidewall between the upper end and lower end defines a longitudinal axis I of fluid filtration apparatus 1.

Housing 5 may be shaped in any way. In one embodiment, housing 5 is shaped in a way that is amenable to injection molding. Housing 5, and more particularly a cross-section of lumen 7 taken in a plane orthogonal to longitudinal axis I of housing 5, is shaped so as to receivingly accommodate a frame 30 (as described further below) that is rotatably supported within lumen 7 and rotatable relative to housing 5 (about an axis of rotation that may be coincident with longitudinal axis I).

In one embodiment, housing 5 is cylindrical or substantially cylindrical. In one embodiment, lumen 7 of housing 5 is cylindrical or substantially cylindrical.

In one embodiment, housing 5 (or at least on sidewall thereof) comprises an outer surface and an inner surface (e.g. a lumen-facing surface). In one embodiment, the outer surface of at least one sidewall 10 of housing 5 tapers from an upper end of the housing 5 to a lower end of the housing 5. In one embodiment, the lumen-facing surface of at least one sidewall 10 tapers from an upper end of the housing 5 to a lower end of the housing 5. In one embodiment, both the outer surface and the lumen-facing surface are tapered. In one embodiment, both the outer surface and the lumen-facing surface taper from an upper end of the housing 5 to a lower end of the housing 5.

If present, a taper (or draft angle) of the outer surface and/or the lumen-facing surface of at least one sidewall 10 is less than 10 degrees, or is less than 9 degrees, or is less than 8 degrees, or is less than 7 degrees, or is less than 6 degrees, or is less than 5 degrees, or is less than 4 degrees, or is less than 3 degrees, or is less than 2 degrees, or is about 1 degree (±0.5 degrees) or less.

Housing 5 may be made or formed of any material. In one embodiment, the material is biocompatible. In one embodiment, housing 5 is made of a polymer or a plastic, such as any type of polymer or plastic used to form conventional cell culture vessels or apparatus. In one embodiment, housing 5 is sterilizable, such as by contact with alcohol, irradiation, or autoclave.

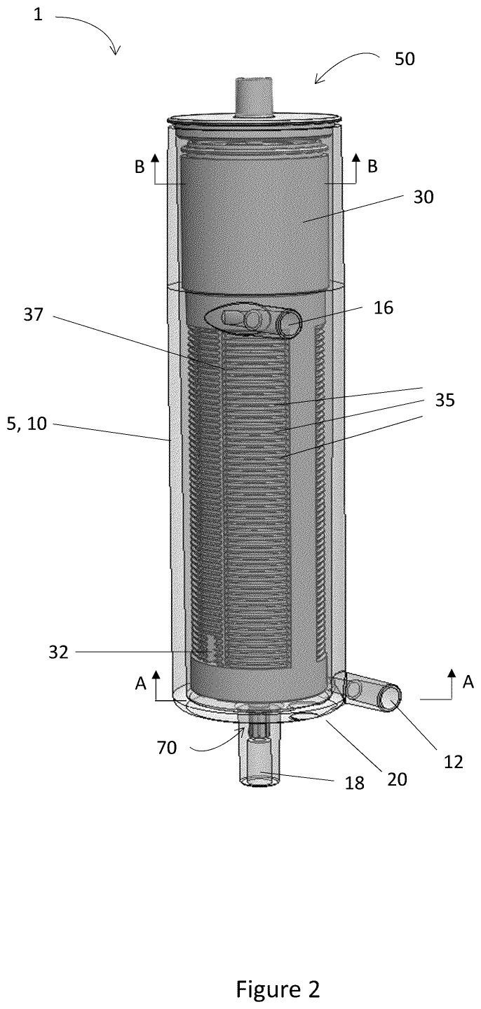

Fluid filtration apparatus 1 further comprises a plurality of ports (FIGS. 1 and 2). In one embodiment, fluid filtration apparatus 1 comprises an inlet 12 and an outlet 16 through housing 5. In one embodiment, one or both of inlet 12 and outlet 16 are configured in (or through) at least one sidewall 10. Each of inlet 12 and outlet 16 are in fluid communication with lumen 7.

In one embodiment, inlet 12 and outlet 16 are integral with housing 5, and comprise a bore therethrough. In one embodiment, inlet 12 and outlet 16 are connected to or continuous with respective bores through at least one sidewall 10.

In one embodiment, inlet 12 (or bore therethrough) is tangential to at least one sidewall 10. In one embodiment, inlet 12 (or bore therethrough) is non-tangential to at least one sidewall 10. In one embodiment, outlet 16 (or bore therethrough) is tangential to at least one sidewall 10. In one embodiment, outlet 16 (or bore therethrough) is non-tangential to at least one sidewall 10. In one embodiment, both inlet 12 and outlet 16 (or bores therethrough) are tangential to at least one sidewall 10. In one embodiment, both inlet 12 and outlet 16 (or bores therethrough) are non-tangential to at least one sidewall 10.

Fluid filtration apparatus 1 may also comprise a second outlet 18 (e.g. a waste port) through housing 5 (FIGS. 1, 2 and 7). In one embodiment, second outlet 18 is configured in a bottom wall 20 of housing 5. In one embodiment, second outlet 18 is integral with housing 5. In one embodiment, second outlet 18 is connected to or continuous with a bore through housing 5, such as through bottom wall 20.

The position of inlet 12, first outlet 16, and second outlet 18 (if present) in/through housing 5 is flexible. In one embodiment, at least inlet 12 and first outlet 16 are spaced apart, such as along longitudinal axis I of housing 5, and more particularly of sidewall 10. In one embodiment, each of inlet 12, first outlet 16, and second outlet 18 are spaced apart, such as along longitudinal axis I of housing 5, and more particularly of sidewall 10.

In one embodiment, inlet 12 is configured through housing 5 at a position relatively higher along longitudinal axis I than first outlet 16. In such embodiment, inlet 12 may be configured through housing 5 (e.g. sidewall 10) at or near an upper end of housing 5, and first outlet 16 may be configured through housing 5 (e.g. sidewall 10) at or near a lower end of housing 5.

In one embodiment, inlet 12 is configured through housing 5 at a position relatively lower along longitudinal axis I than first outlet 16. In such embodiment, inlet 12 may be configured through housing 5 (e.g. sidewall 10) at or near a lower end of housing 5 and first outlet 16 may be configured through housing 5 (e.g. sidewall 10) at or near an upper end of housing 5.

Regardless of which of inlet 12 or first outlet 16 is positioned relatively lower than the other (along longitudinal axis I), the lower of the two may be configured at a lower end of housing 5, and substantially in-line with a lumen-facing surface of bottom wall 20.

In embodiments of fluid filtration apparatus 1 comprising second outlet 18, the second outlet 18 may be configured through housing 5, and more specifically may be configured through bottom wall 20 of housing 5. Thus, one of inlet 12 and first outlet 16 may be positioned intermediate the other and second outlet 18.

In embodiments where one or both of inlet 12 and first outlet 16 are positioned (through at least one sidewall 10) at or near an upper end of housing 5, it may be important to maintain a sufficient amount of clearance between the position of inlet 12 and/or first outlet 16 and the top of housing 5. For example, and as further described below, mechanical components for rotating frame 30 and/or rotatably connecting housing 5 and a frame 30 that is rotatable within lumen 7.

Since inlet 12, first outlet 16 and second outlet 18 are configured to transmit fluid therethrough, they may thus be mateable with respective ends of tubes or other conduits through which the fluid flows. In one embodiment, one or more of inlet 12, first outlet 16 and second outlet 18 comprise an adapter or connector that mediates mating or connection with respective ends of tubing or conduit.

In one embodiment, one or more of inlet 12, first outlet 16 and second outlet 18 may be mateable with respective ends of tubing or conduit, such as by inserting or sliding an end of tubing or conduit around a respective mouth of inlet 12, first outlet 16 and second outlet 18. In one embodiment, one or more of inlet 12, first outlet 16 and second outlet 18 may be mateable with a respective end of tubing or conduit, such as by means of an adhesive or another type of fastener (e.g. luer lock, threading, etc).

In one embodiment, inlet 12 may include a pressure sensor to measure fluid pressure at the inlet 12. In one embodiment, first outlet 16 may include a pressure sensor to measure fluid pressure at the first outlet 16. In one embodiment, second outlet 18 may include a pressure sensor to measure fluid pressure at the second outlet 18.

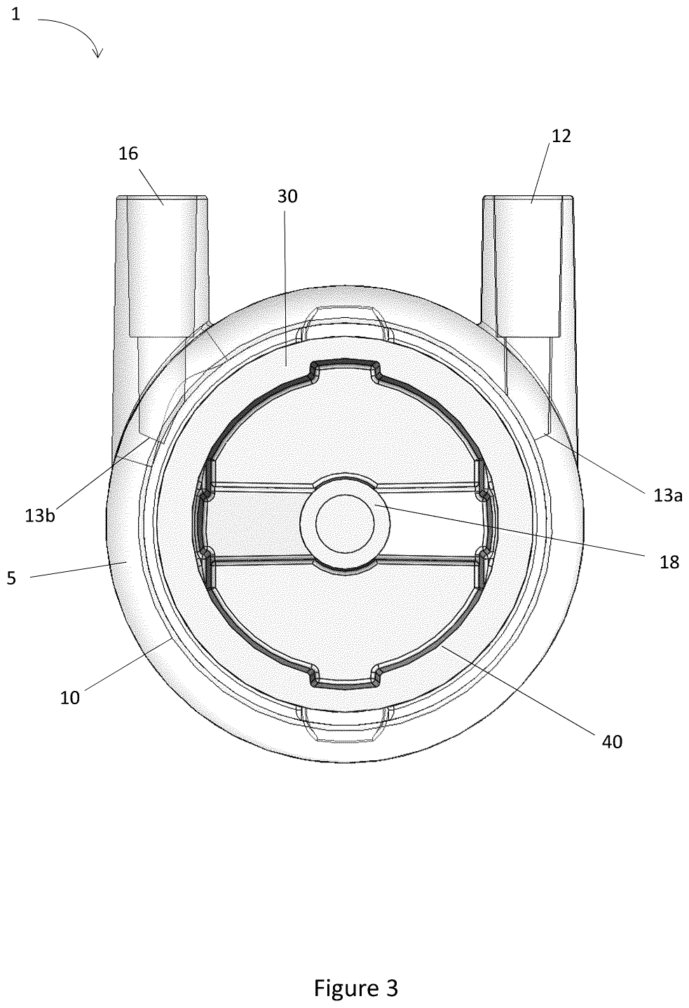

Fluid filtration apparatus 1 may further comprise a baffle 13a configured in at least inlet 12 (FIG. 3). Preferably, baffle 13a may be configured at an opening of inlet 12 into lumen 7. Baffle 13a may be disposed in and/or integral with at least one sidewall 10 of housing 5, and more particularly may be disposed within a bore extending through at least one sidewall 10.

In one embodiment, fluid filtration apparatus 1 may further comprise a baffle 13b configured in first outlet 16. Preferably, baffle 13b may be configured at an opening of first outlet 16 into lumen 7. Baffle 13b may be disposed in and/or integral with at least one sidewall 10 of housing 5, and more particularly may be disposed within a bore extending through at least one sidewall 10. In one embodiment, both inlet 12 and first outlet 16 comprise baffles 13a and 13b.

In one embodiment, a tangential inlet 12 directs a fluid into housing 5, and more particularly into lumen 7 thereof. In one embodiment, a non-tangential inlet 12 directs a fluid into housing 5, and more particularly into lumen 7 thereof. In such embodiments, baffle 13a may facilitate the dispersion of fluids (and particulates suspended therein) in the lumen 7 and/or the mixture of a fluid to be processed using fluid filtration apparatus 1.

Fluid filtration apparatus 1 further comprises a frame 30 supported within lumen 7 of housing 5 (FIGS. 1 and 2). Frame 30 may also be referred to herein as a rotor, or a spinner, or a rotary spinner, and these terms may be used interchangeably with the term “frame”. Thus, in one embodiment, frame 30 is rotatably supported within lumen 7 and is rotatable relative to housing 5. In one embodiment, frame 30 is rotatable in a clockwise and/or in an anti-clockwise direction.

Frame 30 may be made or formed of any material. In one embodiment, the material is biocompatible. In one embodiment, frame 30 is made of a polymer or a plastic, such as any type of polymer or plastic used to form conventional cell culture vessels or apparatus. In one embodiment, frame 30 is sterilizable, such as by contact with alcohol, irradiation, or autoclave.

Frame 30 may be shaped in any way. In one embodiment, frame 30 is shaped in a way that is amenable to injection molding or other types of molding. A cross-section of frame 30, taken in a plane orthogonal to longitudinal axis I of housing 5 and frame 30, is shaped so as to be receivingly accommodated within housing 5 (e.g. within lumen 7) and to be rotatable about the longitudinal axis.

In some embodiments housing 5 is cylindrical or substantially cylindrical, and frame 30 is cylindrical or substantially cylindrical. Thus, lumen 7 and frame 30 will have a diameter (or major axis) taken in a plane orthogonal to longitudinal axis I. In such embodiments, a diameter (or major axis) of frame 30 is less than a diameter of lumen 7. In one embodiment, a diameter of frame 30 is concentric with a diameter of at least one sidewall 10.

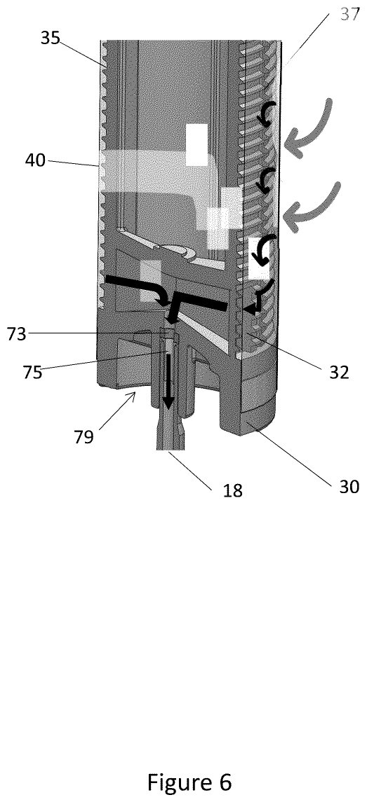

In one embodiment, frame 30 comprises at least one bore 32 therethrough (FIG. 6). In one embodiment, frame 30 comprises more than one bore 32 therethrough. Bore(s) 32 may converge and be in fluid communication with second outlet 18. Together, bore(s) 32 and second outlet 18 convey fluid and/or filtrate out of fluid filtration apparatus 1, after having been introduced into fluid filtration apparatus 1 via inlet 12 and passed through a first porous member, as further described below.

At least one bore 32 may be positioned at any point along a length of frame 30 (as defined by longitudinal axis I). Preferably, at least one bore 32 is positioned at or near a base or a lower end of frame 30, where filtrate may settle after having passed through a first porous member supported on frame 30. In one embodiment, at least one bore 32 is positioned near a lower end of frame 30, but spaced apart from a base of frame 30 by a bearing surface about a perimeter of frame 30. Such bearing surface about a perimeter of frame 30 (between the lower end of frame 30 and at least one bore 32) may be important for securing a first porous member 40 to frame 30, as further described below.

In one embodiment, frame 30 may comprise two bores 32 opposed (e.g. diametrically) to one another, and the bores converge within frame 30 to create a channel in fluid communication with second outlet 18, or in fluid communication with a conduit leading to second outlet 18.

Frame 30 may further comprise a plurality of spaced-apart latitudinal grooves 34 in a porous member facing surface of frame 30 (e.g. a lumen-facing surface of frame 30) (FIGS. 1, 2, and 6). In one embodiment, plurality of spaced-apart latitudinal grooves 34 are parallel or substantially parallel to one another. Plurality of spaced-apart latitudinal grooves 34 each form a trough in a lumen-facing surface of frame 30. Thus, plurality of spaced-apart latitudinal grooves 34 may be intervened by a plurality of spaced apart latitudinal ridges 35. In one embodiment, plurality of spaced apart latitudinal ridges 35 are parallel or substantially parallel to one another (and to plurality of spaced-apart latitudinal grooves 34).

In one embodiment, some or all of plurality of spaced-apart latitudinal grooves 34 (and plurality of spaced apart latitudinal ridges 35) are disposed completely about a lumen-facing surface of frame 30. In one embodiment, some or all of plurality of spaced-apart latitudinal grooves 34 (and plurality of spaced apart latitudinal ridges 35) are not disposed completely about a lumen-facing surface of frame 30.

Frame 30 may further comprise a plurality of spaced-apart longitudinal grooves 37 in a porous member facing surface of frame 30 (e.g. a lumen-facing surface of frame 30) (FIGS. 1, 2, and 6). In one embodiment, plurality of spaced-apart longitudinal grooves 37 are parallel or substantially parallel to one another. Plurality of spaced-apart longitudinal grooves 37 each form a trough in a lumen-facing surface of frame 30. Thus, plurality of spaced-apart longitudinal grooves 37 may be intervened by a plurality of spaced apart longitudinal ridges 38. In one embodiment, plurality of spaced apart longitudinal ridges 38 are parallel or substantially parallel to one another (and to plurality of spaced-apart longitudinal grooves 37).

In one embodiment, some or all of plurality of spaced-apart longitudinal grooves 37 are disposed parallel to longitudinal axis I of frame 30 (and housing 5). In one embodiment, plurality of spaced-apart longitudinal grooves 37 are formed substantially along the entirety of frame 30. In one embodiment, a porous-membrane supporting surface of frame 30 may be defined by the longitudinal extent of the plurality of spaced-apart latitudinal grooves 34 (and plurality of spaced apart latitudinal ridges 35). In one embodiment, a porous membrane supporting surface of frame 30 may be defined by plurality of latitudinal ridges 35 and/or plurality of longitudinal ridges 38.

In one embodiment, plurality of spaced-apart longitudinal grooves 37 interconnect plurality of spaced-apart latitudinal grooves 34, thereby creating a network of grooves/troughs in a lumen-facing surface (and membrane supporting surface) of frame 30.

Plurality of spaced-apart latitudinal grooves 34 may be any width, provided that they are sufficiently wide (and deep) to accommodate a cell, or any particulates in a filtrate having passed through first porous membrane 40. For example, a width (and depth) of plurality of spaced-apart latitudinal grooves 34 may be between about 2 μm to 5 mm, or between about 3 μm to 4 mm, or between about 5 μm to 3 mm.

Similarly, plurality of spaced-apart longitudinal grooves 37 may be any width, provided that they are sufficiently wide (and deep) to accommodate a cell, or any particulates in a filtrate having passed through first porous membrane 40. For example, a width (and depth) of plurality of spaced-apart longitudinal grooves 37 may be between about 2 μm to 5 mm, or between about 3 μm to 4 mm, or between about 5 μm to 3 mm.

In one embodiment, a width of each of the plurality of spaced-apart latitudinal grooves 34 is equal. In one embodiment, a width of each of the plurality of spaced-apart latitudinal grooves 34 is not equal. In a non-limiting example, a width of each of the plurality of latitudinal grooves 34 may increase from a lower end to an upper end of frame 30. In a different non-limiting example, a width of each of the plurality of latitudinal grooves 34 may decrease from a lower end to an upper end of frame 30.

Fluid filtration apparatus 1 further comprises a first porous member 40 secured to or about frame 30 (FIG. 1). In one embodiment, fluid filtration apparatus 1 comprises only one porous member. In one embodiment, fluid filtration apparatus 1 comprises a plurality of porous members, such as two porous members that may be overlain. In embodiments comprising a plurality of porous members, an inner porous member may be sandwiched between frame 30 and an outer porous member (in an overlapping fashion).

First porous member 40 (and additional porous members) is not particularly limited, provided it does not tear or become otherwise compromised (e.g. weakened, stretched, or a reduction in performance) under the conditions in which fluid filtration apparatus 1 operates (e.g. pressure and pressure change forces).

Porous member(s) may be one or more of fibrous mesh membranes, cast membranes, track etch membranes, or other types of membranes known to those of skill in the art. In one embodiment, first porous member 40 is a track etch membrane. In one embodiment, first porous member 40 is made of a polymer, such as polyester or polycarbonate.

Since pressure differences may arise on either side of first porous membrane 40 during operation of fluid filtration apparatus 1, first porous member may withstand such pressure drops. In one embodiment, attachment means for securing first porous member 40 to frame 30 may withstand pressure differences on either side of first porous membrane 40.

First porous member 40 (and additional porous members) may also be constrained by the pore size. In general, the pore size will depend on the particular application of fluid filtration apparatus 1 and the nature of the fluid to be processed. Thus, pore size may be carefully selected depending on the nature of the particulates in the fluid intended to pass through first porous member 40. As an example, at least two populations of particles (based on average diameter) comprised in a fluid may be fractionated by selecting a pore size of first porous member 40 that only permits the relatively smaller particle population(s) to pass through. As a more specific, not limiting, example, the pores may be sized to allow small filtrate (e.g., platelets, microparticles, etc.) to pass through first porous member 40 (and additional porous members), while the desired retentate residue does not (e.g., cells, such as white blood cells). In this way, it may be possible to fractionate differently-sized particles or cells in a fluid by selecting a pore size that excludes at least some of the particles or cells.

In terms of pore size, first porous member may comprise a plurality of pores having a nominal pore size below 10 μm. In one embodiment, first porous member comprises a plurality of pores having a nominal pore size of about 9 μm. In one embodiment, first porous member comprises a plurality of pores having a nominal pore size of about 8 μm. In one embodiment, first porous member comprises a plurality of pores having a nominal pore size of about 7 μm. In one embodiment, first porous member comprises a plurality of pores having a nominal pore size of about 6 μm. In one embodiment, first porous member comprises a plurality of pores having a nominal pore size of about 5 μm. In one embodiment, first porous member comprises a plurality of pores having a nominal pore size of about 4 μm. In one embodiment, first porous member comprises a plurality of pores having a nominal pore size of about 3 μm. In one embodiment, first porous member comprises a plurality of pores having a nominal pore size of about 2 μm. In one embodiment, first porous member comprises a plurality of pores having a nominal pore size in the range of 2 μm to 6 μm.

In one embodiment, first porous member 40 (and additional porous members) are also constrained by the pore density. In one embodiment, the pore density may be in the range of 1×10⁵ pores/cm² to 1×10⁶ pores/cm². In one embodiment, the pore density may be in the range of 2×10⁵ pores/cm² to 8×10⁵ pores/cm². In one embodiment, the pore density may be in the range of 3×10⁵ pores/cm² to 6×10⁵ pores/cm². In one embodiment, the pore density may be about 4×10⁵ pores/cm²±1×10⁵.

In one embodiment, first porous member 40 is secured to frame 30 using a biocompatible adhesive 42. In one embodiment, adhesive 42 is a pressure sensitive adhesive. In one embodiment, adhesive 42, such as a pressure sensitive adhesive, may be applied about the perimeter of first porous member 40. In one embodiment, adhesive 42, such as a pressure sensitive adhesive, may be applied to some or all of plurality of spaced-apart latitudinal ridges 35 and/or plurality of spaced-apart longitudinal ridges 38 of frame 30.

In one embodiment, first porous member 40 may be supported on a membrane-bearing surface of frame 30, such as against plurality of spaced-apart latitudinal ridges 35 and/or plurality of spaced-apart longitudinal ridges 38. In one embodiment, first porous member 40 is adhered to plurality of spaced-apart latitudinal ridges 35 and/or plurality of spaced-apart longitudinal ridges 38. In one embodiment, first porous member 40 is not adhered to plurality of spaced-apart latitudinal ridges 35 and/or plurality of spaced-apart longitudinal ridges 38, but is taut against plurality of spaced-apart latitudinal ridges 35 and/or plurality of spaced-apart longitudinal ridges 38, and secured at its outermost edges to frame 30 by adhesive 42. As indicated above, bearing surfaces on frame 30, such as about a perimeter of frame 30 at opposed ends thereof and/or running a length of frame 30 in the direction of longitudinal axis I, may facilitate attachment of first porous member 40 to frame 30.

In one embodiment, ends of first porous member 40 do not overlap when secured to or about frame 30. In one embodiment, ends of first porous member 40 overlap when secured to or about frame 30.

As indicated above, frame 30 is rotatable about an axis of rotation (which may be coincident with longitudinal axis I) within housing 5, and more particularly within lumen 7 of housing 5. Thus, there should be sufficient clearance between an extremity of frame 30 and inner sidewall 10 of housing 5, which may be referred to as a shear gap. A typical shear gap may be approximately between 0.1 mm and 10 mm, or between about 0.25 mm and 5 mm, or between about 0.5 mm and 3 mm. In one embodiment, the shear gap may be uniform or substantially uniform along its length (i.e. parallel to the longitudinal axis). In one embodiment, the shear gap may vary along its length (i.e. parallel to the longitudinal axis), for example, the shear gap may (continuously) increase or decrease in a direction from an upper end of frame 30 to a lower end of frame 30.

In one embodiment, a diameter of lumen 7 is larger at an upper end of housing 5 relative to a lower end of housing 5. In such embodiments, a diameter of lumen 7 may gradually/continuously decrease from upper end of housing 5 to lower end of housing 5. In the same or different embodiments, a diameter of frame 30 may be larger at an upper end thereof relative to a lower end thereof, and in such embodiments a diameter of frame 30 may gradually/continuously decrease from upper end thereof to lower end thereof.

In one embodiment, a diameter of lumen 7 is smaller at an upper end of housing 5 relative to a lower end of housing 5. In such embodiments, a diameter of lumen 7 may gradually/continuously increase from upper end of housing 5 to lower end of housing 5. In the same or different embodiments, a diameter of frame 30 may be smaller at an upper end thereof relative to a lower end thereof, and in such embodiments a diameter of frame 30 may gradually/continuously increase from upper end thereof to lower end thereof.

A tapering configuration of at least one sidewall 10, and more specifically a lumen-facing surface of at least one sidewall 10, from the upper end to the lower end of housing 5 may improve filtration efficiency. In one embodiment, a tapering configuration of at least one sidewall 10 may influence fluid flow patterns within lumen 7. For example, a tapering configuration and resultant (continuous) change in shear gap width along the direction of longitudinal axis I may result in progressively longer (or shorter) axial wave lengths of a toroidal vortex and/or a Taylor vortex.

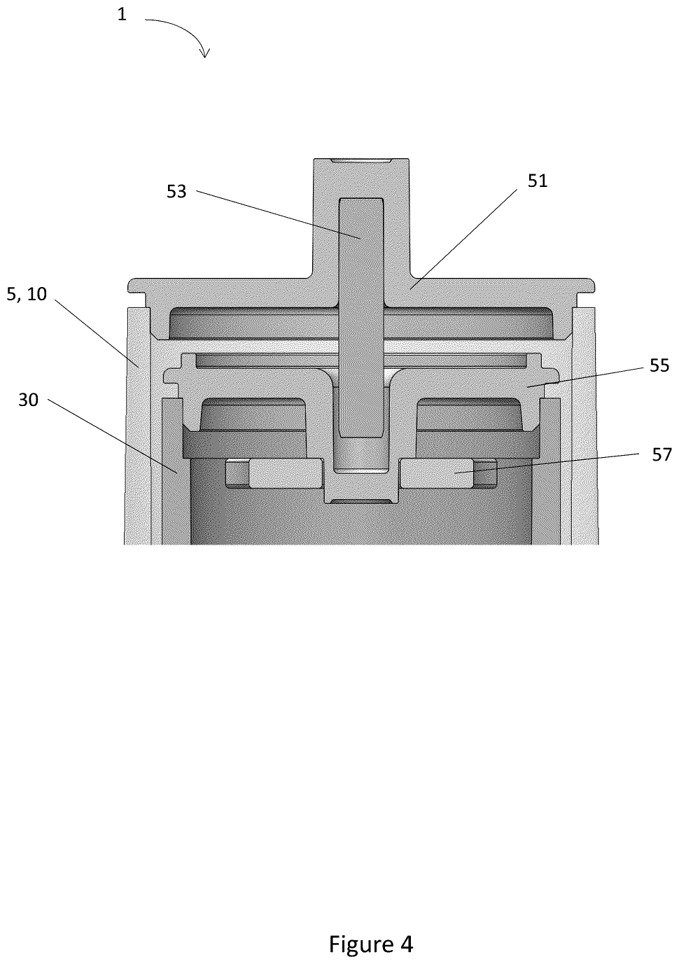

With reference to FIGS. 4-7, fluid filtration apparatus 1 may further comprise an upper assembly 50 and a lower assembly 70 (further described below) that cooperate respectively with the upper end of housing and the lower end of housing to rotatably support frame 30 in lumen 7. In one embodiment, upper assembly 50 comprises a bearing. In one embodiment, lower assembly 70 comprises a bearing. In one embodiment, only one of upper assembly 50 and lower assembly 70 comprise a bearing. In one embodiment, both upper assembly 50 and lower assembly 70 comprise a bearing. In embodiments where upper assembly 50 comprises a bearing it may be referred to as an upper bearinged assembly 50. In embodiments where lower assembly 70 comprises a bearing it may be referred to as a lower bearinged assembly 70

Upper assembly 50 may comprise numerous components that cooperate to seal an upper end of housing 5. Upper (bearinged) assembly 50, and the components thereof, may also cooperate to support an upper end of frame 30 in rotatable relationship relative to an upper end of housing 5. Thus, in one embodiment, upper (bearinged) assembly 50 rotatably connects frame 30, at an upper end thereof, and an upper end of housing 5.

In one embodiment, upper (bearinged) assembly 50 comprises a housing cap 51.

Housing cap 51 is mounted or otherwise attached to housing 5. In one embodiment, housing cap 51 is mounted to housing 5 by means of an adhesive, or another type of fastening means such as by threads or screws. In one embodiment, housing cap 51 is mounted or attached to housing 5 by an interference fit or press fit.

In one embodiment, housing cap 51 seals an upper end of housing 5, such as by being slidably received or threaded into lumen 7. In one embodiment, housing cap 51 comprises a collar descending from an underside thereof that is dimensioned to be received within lumen 7 of housing 5. In one embodiment, a flange on housing cap 51 limits a distance it may be received into lumen 7.

In one embodiment, upper (bearinged) assembly 50 comprises an upper bearing 53. Upper bearing 53 may be connected to housing cap 51 at an underside thereof. Upper bearing 53 may be adhered to housing cap 51, may be connected thereto by interference fit, or attached by any other means. In one embodiment, upper bearing 53 mediates rotatable coupling of frame 30, or more directly a frame cap 55 thereof, to the stationary or fixed housing cap 51. In addition to mediating the rotation of frame 30 (and frame cap, or rotor cap 55) within housing 5, upper bearing 53 may further align frame 30 (and frame cap, or rotor cap 55) within housing 5 for the controlled rotation of frame 30 (and frame cap, or rotor 55) relative to housing cap 51, about its axis of rotation.

In one embodiment, housing cap 51 and or frame cap 55 is made or formed of any material. In one embodiment, the material is biocompatible. In one embodiment, housing cap 51 and/or frame cap 55 is made of a polymer or a plastic, such as any type of polymer or plastic used to form conventional cell culture vessels or apparatus. In one embodiment, housing cap 51 and/or frame cap 55 is sterilizable, such as by contact with alcohol, irradiation, or autoclave.

In one embodiment, frame cap 55 is mounted or connected to frame 30 by means of an adhesive. In one embodiment, frame cap 55 is threaded into frame 30. In one embodiment, frame 30 is mounted to frame cap 55 by means of screws and nuts, rivets, or any other fastening means. Thus, frame cap 55 (and frame 30) may be rotatably supported in upper (bearinged) assembly 50, and more specifically is secured to upper bearing 53 in a way that permits rotation of frame 30 relative to housing 5.

In one embodiment, frame cap 55 seals an upper end of frame 30, such as by being slidably received or threaded into an upper end of frame 30. In one embodiment, frame cap 55 comprises a collar descending from an underside thereof that is dimensioned to be received within an upper end of frame 30. In one embodiment, a flange on frame cap 55 limits a distance it may be received into an upper end of frame 30.

Upper (bearinged) assembly 50 may further comprise drive means to rotate frame (or rotor) 30 within housing 5, or means that cooperate with drive means external to housing 5 to rotate frame (or rotor) 30 within housing 5.

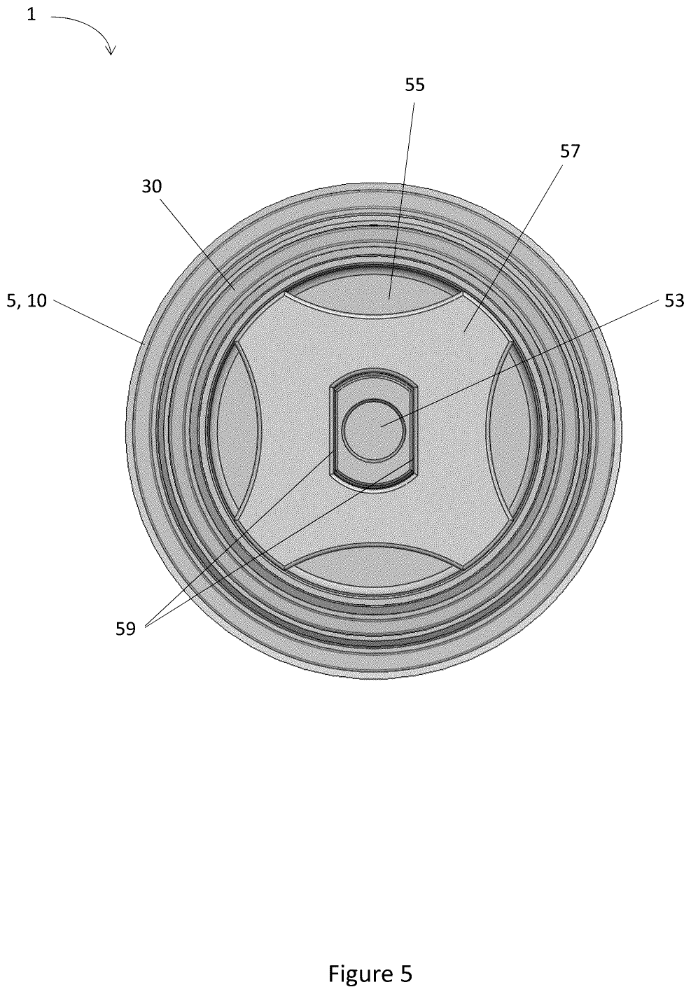

In one embodiment, upper (bearinged) assembly 50 further comprises a magnetically-responsive member 57 arranged in frame 30, preferably in an upper end thereof (FIGS. 4 and 5). Magnetically-responsive member 57 may be embedded within frame or rotor cap 55 or it may be a separate component of upper (bearinged) assembly 50, but in a fixed relationship therewith (e.g. not rotatable relative to frame 30 or frame cap 55). In one embodiment, magnetically-responsive member 57 is mounted or adhered to an underside of frame cap 55. In one embodiment, magnetically-responsive member 57 is received within frame 30 before frame cap 55 is secured to frame 30.

Magnetically-responsive member 57 may comprise or may be made from a permanent magnet. Thus, magnetically-responsive member 57 may indirectly facilitate rotation of frame 30 under the influence of an appropriate magnetic field, such as an oscillating, intermittent or rotating magnetic field. In one embodiment, magnetically-responsive member comprises points or projections that are interspersed by voids or non-magnetically responsive segments (when viewed in the plane orthogonal to longitudinal axis I).

Magnetically-responsive member 57, including one or more magnetic or magnetically-responsive segments interspersed by non-magnetically-responsive or -susceptible segments, may be manufactured into a desired shape by punching, machining, or otherwise. In one embodiment, magnetically-responsive member 57 is star- or gear-shaped.

In one embodiment, rotation of frame or rotor 30 within housing 5 is effected by the responsiveness of magnetically-responsive member 57 to an externally applied magnetic field. Thus, manipulating the externally applied magnetic field will cause magnetically-responsive member 57 (and frame 30) to rotate about axis of rotation.

Magnetically-responsive member 57 may further comprise a feature that mediates its coupling to frame 30 or frame cap 55. Thus, magnetically, responsive member 57 and frame 30 or frame cap 55 may be mated via a pair of complementary features.

In one embodiment, magnetically-responsive member 57 further comprises an aperture or depression that mates with a projection of frame 30 (or frame cap 55). The aperture or depression may be any shape, provided that it is complementary and/or mateable or connectable to frame 30 or frame cap 55. In one embodiment, the aperture or depression comprises at least one straight or substantially straight edge. In one embodiment, the aperture or depression comprises at least two straight or substantially straight edges.

In one embodiment, magnetically-responsive member 57 further comprises a projection that mates with an aperture or depression of frame 30 (or frame cap 55). The aperture or depression may be any shape, provided that it is complementary and/or mateable or connectable to magnetically-responsive member 57. In one embodiment, the aperture or depression comprises at least one straight or substantially straight edge. In one embodiment, the aperture or depression comprises at least two straight or substantially straight edges.

At least one or at least two straight of substantially straight edges of aperture or depression (and also in the complementary projection mateable with the aperture or depression), may permit a higher degree of torque to be applied through magnetically-responsive member 57 to frame 30, in comparison to a round or rounded aperture or depression which may undergo slippage under high degrees of torque and/or repeated usage.

In one embodiment, magnetically-responsive member 57 is secured within upper (bearinged) assembly 50, such as by bonding using an adhesive, or by interference or press fit. In one embodiment, magnetically-responsive member 57 is embedded or fixed within a component of upper (bearinged) assembly 50, such as frame cap 55.

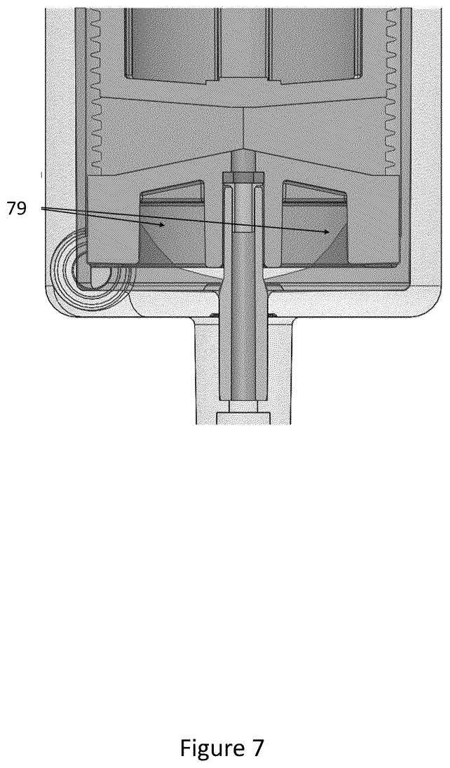

Referring to FIGS. 6 and 7, fluid filtration apparatus 1 may further comprise a lower assembly 70 connecting frame 30 at a lower end thereof and a lower end of housing 5. In one embodiment, lower assembly 70 rotatably connects frame 30 at a lower end thereof and a lower end of housing 5. In one embodiment, a subcomponent, of lower assembly 70 is received within a bore in bottom wall 20, such as may associate with second outlet 18. Thus, the subcomponent, if configured as a conduit may thus intermediate the flow of fluid and any particulates suspended therein through at least one bore 32 of frame 30 and out of fluid filtration apparatus 1 through second outlet 18.

In one embodiment, lower assembly 70 comprises a plurality of subcomponents, including a seal 71. Seal 71 may be made from any material, provided that such material prevents leakage of fluid (and particulates suspended therein) from fluid filtration apparatus 1 other than via second outlet 18. More specifically, seal 71 desirably prevents leakage of fluid (and particulates suspended therein) —having traversed first porous member 40 and entered at least one bore 32—from fluid filtration apparatus 1 other than via second outlet 18. In one embodiment, seal 71 is a gasket, grommet, or the like. In one embodiment, seal 71 is a bead or layer of an inert substance, such as a silicone-based grease.

Lower assembly 70 may further comprise a lower bearing 73. In one embodiment, lower bearing 73 comprises a conduit receivable within a lower end of frame 30 and in fluid communication with at least one bore 32. In one embodiment, lower bearing 73 may be adhered to a lower end of frame 30. In one embodiment, lower bearing 73 may be connected to a lower end of frame 30, such as by interference fit, or attached by any other means. In one embodiment, lower bearing 73 is a conduit in fluid communication with at least one bore 32 of frame 30.

In one embodiment, a side or end of lower bearing 73 may be connected or adhered to housing 5, such as at a bore through bottom wall 20 that associates with second outlet 18. In one embodiment, lower bearing 73 may be connected thereto by interference fit, or attached by any other means.

In one embodiment, lower bearing 73 mediates rotatable alignment of frame 30 within housing 5, such as at second outlet 18. In addition to mediating the rotation of frame 30 within housing 5, lower bearing 73 may cooperate with upper bearing 53 to align frame 30 (and upper assembly 50) relative to second outlet 18 for the controlled rotation of frame 30 within housing 5 about its axis of rotation, which may be coincident with longitudinal axis I.

In one embodiment, lower assembly 70 comprises a bearing support 75. Bearing support 75 may be receivable within housing 5, such as on or within a bore through bottom wall 20 that associates with second outlet 18. In one embodiment, bearing support 75 may be adhered to or in second outlet 18. In one embodiment, bearing support 75 may be connected to or in second outlet 18, such as by interference fit, an adhesive, or attached by any other means. In one embodiment, bearing support 75 comprises a conduit in communication with lower bearing 73 (and more specifically a conduit therethrough) and at least one bore 32 of frame 30. In one embodiment, bearing support 75 provides a seat or interface for lower bearing 73 to be supported within housing 5, such as on or within second outlet 18.

In one embodiment, seal 71 may cooperate with other features, such as features of housing 5 and/or frame 30 and/or lower assembly 70, to prevent or limit leakage of fluid (and particulates suspended therein) from fluid filtration apparatus 1 other than via second outlet 18.

In one embodiment, a bottom side of frame 30 comprises an annular recess concentric with lower bearing 73 and/or bearing support 73 (FIGS. 6 and 7).

In operation, oscillation of a magnetic field or rotation of a magnetic field external of fluid filtration apparatus 1 may cause the rotation of frame 30 about its rotational axis (which may be coincident with longitudinal axis I, as may be defined by a direction between upper assembly 50 and lower assembly 70, or more specifically by upper bearing 53 and lower bearing 73) by influencing movement of magnetically-responsive member 57. Thus, a rotation of frame 30 in either a clockwise or anti-clockwise direction may be effected by a magnetic field. As fluid (and any particulates suspended therein) is introduced into housing 5 via inlet 12 and frame 30 rotates within housing 5, toroidal or Taylor vortices may emerge in the shear gap between frame 30 and inner sidewall 10. A portion of fluid and certain particulates suspended therein (e.g. a filtrate) may traverse first porous member 40 while the remainder of the fluid and remaining filtrates (e.g. a residue or retentate) exits fluid filtration apparatus 1 via first outlet 16. With reference to FIG. 6, plurality of spaced-apart latitudinal 34 and longitudinal grooves 37 cooperate to receive a filtrate having passed through at least first porous member 40 and shuttle the filtrate toward second outlet 18 via at least one bore 32 in frame 30.

Thus, fluid filtration apparatus 1 as described herein may be used in methods of filtering or processing a fluid comprising particulates. In various embodiments, the methods may comprise one or more of: introducing via inlet 12 a fluid flow into fluid filtration apparatus 1, and more particularly into lumen 7, and still more particularly into the shear gap between at least one sidewall 10 and first porous member 40; rotating frame 30, such as by oscillating or rotating a (external) magnetic field to influence movement of magnetically-responsive member 57; passing a filtrate (and size-limited particulate matter) through first porous member 40; shuttling the filtrate toward second outlet 18 via at least one bore 32 in frame 30; and clearing a retentate or residue (not having passed through first porous member) from lumen 7 (or more specifically from the shear gap) via first outlet 16. In one embodiment, rotating the frame (at a desired rate) creates Taylor and/or toroidal vortices in a shear gap between frame 30 and housing 5 (more specifically at least one inner sidewall thereof), and the axial wavelength of the Taylor and/or toroidal vortex may widen or narrow (in the direction of longitudinal axis I) depending on factors, including a tapering of inner sidewall of housing and/or a dimensions/widths of plurality of longitudinal grooves, as described above.

In one embodiment, as fluid is introduced via inlet 12 an air bubble or pocket may form within annular recess 79 of fluid filtration apparatus 1 or methods of using same. In one embodiment, the air bubble or pocket within annular recess 79 may assume a generally funnel or hemispherical shape when the frame 30 is rotated and the fluid is centrifugally pushed/pulled away from an axis of rotation of frame 30 (e.g. longitudinal axis I). Thus, the air bubble or pocket may cooperate with other features of frame 30 and housing 5 (e.g. lower assembly) to prevent or limit leakage of fluid (and particulates suspended therein) from fluid filtration apparatus 1 other than via second outlet 18.

In one embodiment, whole blood may be filtered or processed using fluid filtration apparatus 1 to separate or substantially separate red blood cells and/or platelets and/or other similarly-sized (or smaller) particulate matter from lymphocytes and/or other similarly-sized (or larger) cells or particulate matter. In one embodiment, an apheresis blood sample may be filtered or processed using fluid filtration apparatus 1 to separate or substantially separate platelets and/or other similarly-sized (or smaller) particulate matter in the plasma from lymphocytes and/or other similarly-sized (or larger) cells or particulate matter.

In one embodiment, fluid filtration apparatus 1 may be comprised in a cell separation system that may further fractionate, in a separate cell separation vessel or container, a filtrate having passed through first porous member and effluxed via second outlet 18, or a retentate or residue having vacated lumen 7 via first outlet 16. In one embodiment, the separate cell separation vessel or container is a flask or a bag. In one embodiment, a system may further comprise tubing connecting the cell separation vessel or container to reservoirs comprising cell separation reagents, such as (para) magnetic or magnetically-susceptible particles and antibody compositions that intermediate the attachment of target cells to the particles. In one embodiment, a cell separation system may further comprise a magnet, such as a planar magnet array, to effect the immunomagnetic separation of target cells. In one embodiment, tubing may further connect the cell separation vessel or container to a collection receptacle, for containing the target cells (in the case of positive cell selection) or off-target cells (in the case of negative cell selection).

### Methods

In one aspect, this disclosure relates to a method of manufacturing a spinning filter apparatus (e.g. a fluid filtration apparatus), as described above.

A method of manufacturing a fluid filtration apparatus (as described above) comprises molding various components incorporating one or more of the advantageous features described above, and assembling the components into a fluid filtration apparatus of this disclosure. Thus, any feature or embodiment described above in respect of a fluid filtration apparatus, may likewise be introduced or formed in the process of manufacturing and/or assembling fluid filtration apparatus, as further described below.

The methods may comprise molding a housing and molding a frame. Frame and housing may be molded of any material amenable to being placed within a mold, such as in a liquid or semi-solid form that subsequently solidifies into a desired shape or structure while in the mold. In one embodiment, frame and housing are made of a polymer. In one embodiment, frame and housing are made of a plastic. In one embodiment, frame and housing are made of the same polymer or plastic. In one embodiment, frame and housing are made of different polymers or plastics.

In one embodiment, a frame and a housing are made by the same method of molding. In one embodiment, a frame and a housing are made by different methods of molding. In one embodiment, a frame and/or a housing are made by injection molding.

A molded housing may comprise a lumen bounded by at least one sidewall. Molded housing (and lumen) may comprise an upper end and a lower end connected by at least one sidewall. Thus, the upper end and the lower end may define a longitudinal axis of the molded housing. In one embodiment, the longitudinal axis passes through the lumen. In one embodiment, the longitudinal axis passes through a center point of the lumen, and may thus be coincident with an axis of rotation of frame 30 within lumen 7.

Molded housing (and lumen) and/or frame 30 may be cylindrical or substantially cylindrical. In embodiments of a substantially cylindrical housing, a lumen-facing surface of the at least one sidewall tapers along the longitudinal axis thereof. In one embodiment, at least one sidewall tapers from the upper end of the housing toward the lower end of the housing. In one embodiment, at least one sidewall tapers from the lower end of the housing toward the upper end of the housing. Regardless, the taper may be 10 degrees or less, 9 degrees or less, 8 degrees or less, 7 degrees or less, 6 degrees or less, 5 degrees or less, 4 degrees or less, 3 degrees or less, 2 degrees or less, or about 1 degree (±0.5 degrees) or less.

A tapered or tapering sidewall may facilitate the manufacturing process (e.g. molding processes, such as injection molding). A tapered or tapering sidewall may influence resultant toroidal and/or Taylor vortices within the fluid filtration apparatus, when the frame is rotated in the housing. A tapered or tapering sidewall may improve performance of a fluid filtration process, such as by altering a wavelength of toroidal and/or Taylor vortices.

In one embodiment, one of upper end or lower end of housing is open, and is dimensioned to receive a molded frame through the opening into a lumen of housing. In one embodiment, upper end is open and bottom end comprises a closed bottom wall or a substantially closed bottom wall. In one embodiment, bottom end is open and upper end comprises a closed top wall or a substantially closed top wall. In a preferred embodiment, bottom end comprises a closed bottom wall or a substantially closed bottom wall.

In embodiments of a molded housing comprising a closed or substantially closed top or bottom wall, an outlet (e.g. a second outlet as described above) may be molded therein (e.g. integral with bottom wall) and/or a bore may be molded therethrough.

An outlet and/or bore through closed or substantially closed top or bottom wall may comprise a shoulder or seat that helps rotatably secure frame within lumen, such as by accommodating one or more components of upper assembly or lower assembly, as described above. In one embodiment, the second outlet cooperates to define the longitudinal axis through the lumen, and also an axis of rotation of the frame within housing. Thus, in certain embodiments described herein, the axis of rotation and the longitudinal axis may be the same axis.

Molded housing may further comprise one or both of an inlet and a first outlet. Inlet and/or first outlet will be in fluid communication with lumen. Thus, inlet and/or first outlet may at minimum comprise a bore through at least one sidewall of housing. In one embodiment, while comprising a bore therethrough, inlet and/or first outlet are integral with at least one sidewall and comprise features that mediate connection to tubing or a conduit, such as a spigot or a threading.

As described above, inlet may be tangential to at least one sidewall, and more specifically to an inner sidewall (i.e. lumen facing surface) of the housing. In another embodiment, inlet may be non-tangential to at least one sidewall, and more specifically to an inner sidewall (i.e. lumen facing surface) of the housing. Likewise, in certain embodiments first outlet may either be tangential or non-tangential to at least one sidewall, and more specifically to an inner sidewall (i.e. lumen facing surface) of the housing.

In one embodiment, each of the inlet, the first outlet, and the second outlet is in fluid communication with the housing lumen. In one embodiment, each of the inlet, the first outlet, and the second outlet is integral with the housing.

Molding the housing may further comprise configuring at least one baffle in the housing. In one embodiment, a baffle is configured in the inlet. In one embodiment, a baffle is configured in the first outlet. In one embodiment, a baffle is configured in the inlet and the first outlet. Baffle (of one or both of the inlet and the outlet) may be configured within and/or integral with at least one sidewall of housing. In one embodiment, baffle (of one or both of the inlet and the outlet) may be configured at an opening of inlet and/or first outlet into the lumen.

A baffle in the inlet may facilitate mixing of a fluid sample introduced therethrough into a fluid filtration apparatus, particularly a lumen thereof. A baffle in the inlet may improve performance of a fluid filtration process, such as by enhancing mixing of a fluid sample introduced therethrough into a fluid filtration apparatus. A baffle in the inlet may facilitate molding processes, such as injection molding. A baffle in the first outlet may facilitate molding processes, such as injection molding.

In one embodiment, molding a frame comprises forming at least one bore therethrough. In one embodiment, molding a frame comprises forming at least two bores therethrough. In one embodiment, bore(s) through the frame are formed in a lower end thereof. In one embodiment, bore(s) in the frame are opposed and form a channel therethrough.

In one embodiment, molding a frame may comprise defining a plurality of spaced apart latitudinal grooves therein, as described above. In addition or in the alternative, molding a frame may comprise defining a plurality of spaced apart longitudinal grooves therein, as described above. Also as described above, in certain embodiments a plurality of spaced apart latitudinal grooves and a plurality of spaced apart longitudinal grooves intersect, as described above. In one embodiment, the plurality of spaced apart latitudinal grooves and the plurality of spaced apart longitudinal grooves form a network of intersecting grooves that cooperate, lead to, and/or empty into at least one bore through the frame.

A method of manufacturing a fluid filtration apparatus (as described above) may further comprise attaching a porous member to or about the frame. The porous member may be attached to the frame using any means, such as using an adhesive, by sonic welding, or the like. In one embodiment, the porous member may be attached to the frame by a pressure sensitive adhesive.

In one embodiment, a method of manufacturing a fluid filtration apparatus (as described above) comprises applying an adhesive, such as a pressure sensitive adhesive, to one or more frame-contacting faces of the porous member. For example, an adhesive (e.g. a pressure sensitive adhesive) may be applied about or near to the perimeter of a frame-facing side of the porous member. In one embodiment, a method of manufacturing a fluid filtration apparatus (as described above) comprises applying about or near to the perimeter of a frame-facing side of the porous member a strip of adhesive, such as a pressure sensitive adhesive. In one embodiment, the strip of adhesive corresponds to the perimetric dimensions of a frame-facing side of the porous member and/or to one or more frame-contacting faces of the porous member.

A method of manufacturing a fluid filtration apparatus (as described above) may further comprise applying the porous member to the frame before inserting the frame in the lumen. In one embodiment, applying the porous member to the frame comprises rotating the frame about its axis of rotation to set the porous member on the frame. In one embodiment, a molded frame is secured in a lathe or lathe-like instrument and rotated about its axis of rotation to set the porous member on the frame.

A speed the frame is rotated as the porous member is being set thereon is not particularly limited. In one embodiment, the frame is rotated at a rate between about 0.1 rpm and 60 rpm, between about 0.25 rpm and 45 rpm, between about 0.5 rpm and 30 rpm, or between about 0.75 rpm and 15 rpm. In one embodiment, the frame is rotated at a rate between about 0.1 rpm and 10 rpm. In one embodiment, the frame is rotated at a rate above 5 rpm.

A method of manufacturing a fluid filtration apparatus (as described above) may further comprise applying pressure against the porous member and the frame to secure the porous member to the frame. The amount of pressure should be sufficient enough to bring the two components into contact while not so great as to crush or potentially crush the molded frame. Nevertheless, the degree of pressure that the porous member and/or the frame may withstand will depend on the materials used to manufacture each component. In one embodiment, between about 0 to 100 psi may be applied against the porous member and the frame to secure the porous member to the frame.

In one embodiment, a method of manufacturing a fluid filtration apparatus (as described above), may further comprise inserting a molded frame into a lumen of the housing, and rotatably (about its axis of rotation) supporting the frame within the lumen.

In one embodiment, the frame is secured between an upper end of the housing and a lower end of the housing, such as by an upper (bearinged) assembly and a lower assembly (both as described above) that cooperate respectively with the upper end of the housing and the lower end of the housing.

### Interpretation of Terms

While processes, steps or blocks are presented in a given order, alternative examples may perform routines having steps, or employ systems having blocks or steps, in a different order, and some processes or steps or blocks may be deleted, moved, added, subdivided, combined, and/or modified to provide alternative or sub-combinations. Each of these processes or steps or blocks may be implemented in a variety of different ways. Also, while processes or steps or blocks are at times shown as being performed in series, these processes or steps or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Where a component is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Various features are described herein as being present in “one embodiment” or in some “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A fluid filtration apparatus, comprising:
a housing defining a lumen bounded by at least one sidewall, the housing having an upper end and an opposed lower end;
a non-tangential inlet and a first outlet through the housing, each of the inlet and the first outlet in fluid communication with the lumen;
a second outlet through the housing;
a frame rotatably supported within the lumen and rotatable relative to the housing;
at least one bore through the frame, the at least one bore in fluid communication with the second outlet; and
a first porous member secured to or about the frame.

2. The apparatus of claim 1, further comprising an upper assembly and a lower assembly that cooperate respectively with the upper end of the housing and the lower end of the housing to rotatably support the frame in the lumen.

3. The apparatus of claim 1, wherein a lumen-facing surface of the at least one sidewall tapers from the upper end of the housing to the lower end of the housing by about 5 degrees or less, about 3 degrees or less, or about 1 degree±0.5 degrees.

4. The apparatus of claim 1, further comprising a baffle configured in at least the non-tangential inlet, and preferably configured at an opening of the inlet into the lumen.

5. The apparatus of claim 1, further comprising a plurality of spaced-apart latitudinal grooves in a porous member facing surface of the frame.

6. The apparatus of claim 5, further comprising a plurality of spaced-apart longitudinal grooves in a porous member facing surface of the frame, wherein the plurality of spaced-apart longitudinal grooves interconnect the plurality of spaced-apart latitudinal grooves.

7. The apparatus of claim 6, wherein the plurality of spaced-apart longitudinal grooves and the plurality of spaced-apart latitudinal grooves cooperate to receive a filtrate having passed through the porous member and shuttle the filtrate toward the second outlet via the at least one bore in the frame.

8. The apparatus of claim 1, wherein the frame and/or the housing are cylindrical or substantially cylindrical.

9. The apparatus of claim 2, wherein the upper assembly and the lower assembly define a longitudinal axis through the lumen, and the frame rotates about the longitudinal axis.

10. The apparatus of claim 1, further comprising a magnetically-responsive member fixedly arranged in the frame, preferably in an upper end of the frame.

11. The apparatus of claim 10, wherein the magnetically-responsive member and the frame are mated via a pair of complementary features comprising at least one straight edge.

12. The apparatus of claim 1, wherein the first porous member is secured to the frame by a pressure sensitive adhesive.

13. The apparatus of claim 1, wherein the first porous member is a track etch membrane.

14. The apparatus of claim 1, further comprising a second porous member secured to or about the frame, preferably by a pressure sensitive adhesive.

15. A method of manufacturing a fluid filtration apparatus as described in claim 1, the method comprising:
molding a housing having i) a lumen bounded by at least one sidewall that defines a longitudinal axis, ii) an inlet, iii) a first outlet, and iv) a second outlet, each of the inlet, the first outlet, and the second outlet in fluid communication with the lumen;
configuring at least one baffle in the housing, and preferably in one or both of the inlet and the outlet;
molding a frame;
attaching a porous member to or about the frame; and
securing the frame within the lumen, the frame rotatable within the lumen about an axis of rotation.

16. The method of claim 15, further comprising applying a pressure sensitive adhesive to one or more frame-contacting faces of the porous member.

17. The method of claim 15, further comprising applying the porous member to the frame before inserting the frame in the lumen by rotating the frame about the axis of rotation to set the porous member on the frame.

18. The method of claim 17, further comprising applying pressure against the porous member and the frame to secure the porous member thereto.

19. The method of claim 15, wherein the frame is rotatably supported in the lumen between an upper end of the housing and a lower end of the housing by an upper assembly and a lower assembly that cooperate respectively with the upper end of the housing and the lower end of the housing.

20. The method of claim 15, wherein a lumen-facing surface of the at least one sidewall tapers from the upper end of the housing to the lower end of the housing by about 5 degrees or less, about 3 degrees or less, or about 1 degree.
